(19) United States
(12) Patent Application Publication
(10) Pub. No.: US 2026/0240444 A1
(43) Pub. Date: Aug. 20, 2026

(54) METHOD FOR DETECTING BODY FEATURE PARAMETER BY RADAR BASED ON LOCAL COMPUTING AND APPARATUS THEREFOR

(71) Applicant: Guangdong AI Touch Technology Co., Ltd., Foshan (CN)

(72) Inventors: Fan Zhang, Foshan (CN); Bingshan Li, Foshan (CN); Guocheng Chen, Foshan (CN); Huan Sun, Foshan (CN); Sihan Chen, Foshan (CN); Yining Duan, Foshan (CN); Longbiao He, Foshan (CN)

(21) Appl. No.: 19/074,499

(22) Filed: Mar. 10, 2025

(30) Foreign Application Priority Data
Feb. 18, 2025 (CN) 202510181650.9

(51) Int. Cl. A61B 5/05; A61B 5/00; A61B 5/0205; A61B 5/021; A61B 5/11; G16H 40/67

(52) U.S. Cl. CPC A61B 5/05; A61B 5/0022; A61B 5/0205; A61B 5/7285; G16H 40/67; A61B 5/021; A61B 5/1126; A61B 2562/0257

(57) ABSTRACT

The present disclosure relates to the technical field of radar detecting. Disclosed is a method for detecting a body feature parameter by a radar based on local computing and an apparatus therefor, the method including: performing, via the cloud, a compression distillation operation on a basic computational model to obtain a to-be-deployed model corresponding to the basic computational model; deploying the to-be-deployed model to a device terminal corresponding to the radar to obtain a post-deployment model corresponding to the device terminal; and receiving a target parameter of a target object collected by the radar, and judging whether the target parameter satisfies a condition for computing a body feature parameter, and computing, in response to a judgment result of YES, by the post-deployment model and the target parameter, a target body feature parameter of a target object.

### CROSS REFERENCE TO RELATED APPLICATIONS

The present disclosure claims the priority of Chinese Patent Application No. 2025101816509 filed on Feb. 18, 2025 before CNIPA. All the above are hereby incorporated by reference in their entirety.

### TECHNICAL FIELD

The present disclosure relates to the technical field of radar detecting, and in particular to a method for detecting a body feature parameter by a radar based on local computing and an apparatus therefor.

### BACKGROUND

With the rapid development of smart home and health monitoring technologies, there is a growing demand for dynamic monitoring of human signs in home scenarios. Millimeter-wave radar technology, as a non-contact sensing technology with the advantages of high precision, strong penetration and privacy protection, has gradually become a research hotspot in the field of home health monitoring.

However, the millimeter-wave radar monitoring systems of the prior art usually adopt the architecture of cloud computing, i.e., the device terminal transmits the collected radar signals to the cloud, where the cloud algorithms carry out the reasoning and analysis. The architecture is able to utilize the powerful computing power of the cloud, but it also faces the following disadvantages: 1. Dependence on cloud computing results in devices that cannot work properly without a network connection, limiting their application scenarios; 2. Data transmission to the cloud requires additional communication costs and network delays may affect the real-time data transmission. These problems have seriously constrained the further development and popularization of millimeter-wave radar monitoring systems. It is evident that it is particularly important to provide a method that can improve the ease and efficiency of detecting body feature parameters in millimeter-wave radar monitoring systems.

### SUMMARY

Provided in the present disclosure is a method for detecting a body feature parameter by a radar based on local computing and an apparatus therefor. The offline use of the device terminal is achieved when the radar detects the body feature parameter, thereby improving the real-time nature of the data transmission and the convenience method and the device and thus improving the detection efficiency of the radar on the body feature parameter.

In order to address the aforementioned technical problems, disclosed as a first aspect in the present disclosure is a method for detecting a body feature parameter by a radar based on local computing, the method including:

performing, via the cloud, a compression distillation operation on a pre-trained basic computational model to obtain a to-be-deployed model corresponding to the basic computational model;

deploying the to-be-deployed model to a device terminal corresponding to the radar to obtain a post-deployment model corresponding to the device terminal; and

receiving a target parameter of a target object collected by the radar, and judging whether the target parameter satisfies a preset condition for computing a body feature parameter, and computing, in response to a judgment result of YES, by the post-deployment model and the target parameter, a target body feature parameter of a target object, in which the target parameter of the target object includes a type parameter, a distance parameter, a body movement parameter, and a basic body feature parameter of the target object.

As an optional implementation, in the first aspect of the present disclosure, before the performing, via the cloud, the compression distillation operation on the pre-trained basic computational model to obtain the to-be-deployed model corresponding to the basic computational model, the method further includes:

predicting a detection scenario parameter of the radar and determining, according to the detection scenario parameter, a computational demand parameter of the device terminal corresponding to the radar, wherein the computational demand parameter comprises a computational volume demand parameter and/or a computational result demand parameter;

acquiring a resource utilization parameter of the device terminal, and determining, according to the resource utilization parameter, an operational performance parameter of the device terminal, wherein the operational performance parameter comprises a response duration parameter and/or an operational energy consumption parameter; and

determining, according to the computational demand parameter and the operational performance parameter, the compression distillation demand parameter corresponding to the pre-trained basic computational model;

wherein the performing, via the cloud, a compression distillation operation on a pre-trained basic computational model to obtain a to-be-deployed model corresponding to the basic computational model comprises:

performing, via the cloud and the compression distillation demand parameter, a compression distillation operation on the basic computational model to obtain a to-be-deployed model corresponding to the basic computational model.

As an optional implementation, in the first aspect of the present disclosure, before the deploying the to-be-deployed model to a device terminal corresponding to the radar to obtain a post-deployment model corresponding to the device terminal, the method further includes:

determining a model parameter of the to-be-deployed model, wherein the model parameter comprises at least one of a model size parameter, a model computational volume parameter, a model update manner parameter, and a framework compatibility requirement parameter;

acquiring a deployment environment parameter of the device terminal, wherein the deployment environment parameter comprises a deployment network parameter and/or a deployment system parameter;

determining, according to the model parameter, the deployment environment parameter, and the operational performance parameter, a deployment parameter corresponding to the to-be-deployed model, wherein the deployment parameter comprises a deployment location parameter and/or a deployment time parameter;

wherein the deploying the to-be-deployed model to a device terminal corresponding to the radar to obtain a post-deployment model corresponding to the device terminal includes:

deploying, according to the deployment parameter, the to-be-deployed model to a device terminal corresponding to the radar to obtain a post-deployment model corresponding to the device terminal.

As an optional implementation, in the first aspect of the present disclosure, before the determining, according to the model parameter, the deployment environment parameter, and the operational performance parameter, a deployment parameter corresponding to the to-be-deployed model, the method further includes:

acquiring a first radar parameter of the radar, wherein the first radar parameter comprises at least one of a radar sampling rate parameter, a radar resolution parameter, and a radar data processing requirement parameter;

determining, according to the first radar parameter, the deployment environment parameter, and the operational performance parameter, a deployment impact situation caused by the first radar parameter on the to-be-deployed model, and determining a deployment impact degree value corresponding to the deployment impact situation;

judging whether the deployment impact degree value is greater than or equal to a preset deployment impact level threshold;

determining, in response to a judgment result of NO, according to the model parameter, the deployment environment parameter, and the operational performance parameter, a deployment parameter corresponding to the to-be-deployed model;

determining, in response to a judgment result of yes, according to the first radar parameter, the model parameter, the deployment environment parameter, and the operational performance parameter, a deployment parameter corresponding to the to-be-deployed model.

As an optional implementation, in the first aspect of the present disclosure, the target body feature parameter includes at least a blood pressure parameter, and the body movement parameter includes a body movement type parameter and a body movement amplitude parameter;

wherein the judging whether the target parameter satisfies a preset condition for computing a body feature parameter includes:

judging, according to a type parameter of the target object, whether the type parameter is a preset type parameter;

judging, in response to a judgment of the type parameter being the preset type parameter, according to the distance parameter of the target object, whether the distance parameter is within a preset collecting range;

judging, in response to a judgment of the distance parameter being within the preset collecting range, according to the body movement type parameter of the target object, whether the body movement type parameter is a preset body movement type parameter;

judging, in response to a judgment of the body movement type parameter being the preset body movement type parameter, according to the body movement amplitude parameter of the target object, whether the body movement amplitude parameter is within a preset body movement amplitude range;

confirming, in response to a judgment of the body movement amplitude parameter being within a preset body movement amplitude range, that the target parameter satisfies the preset condition for computing a body feature parameter.

As an optional implementation, in the first aspect of the present disclosure, before the confirming that the target parameter satisfies the preset condition for computing a body feature parameter, the method further includes:

determining a first feature parameter and a second feature parameter corresponding to a basic body feature parameter of the target object, wherein the first feature parameter comprises a collecting time parameter and/or a collecting point number parameter, and wherein the second feature parameter comprises a waveform feature parameter;

judging whether the first feature parameter is greater than or equal to a preset feature parameter threshold;

determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold;

confirming, in response to a judgment of the waveform integrity being greater than or equal to a preset waveform integrity threshold, that the target parameter satisfies the preset condition for computing a body feature parameter.

As an optional implementation, in the first aspect of the present disclosure, the feature parameter threshold includes a collecting time threshold and/or a collecting point number threshold.

The feature parameter threshold is determined by following steps:

acquiring a second radar parameter of the radar, in which the second radar parameter of the radar comprises at least one of a radar sampling rate parameter, a radar resolution parameter, a radar data generating speed parameter, and a communication interface performance parameter of the radar;

determining, according to the second radar parameter, a data transmission situation of the radar, wherein the data transmission situation of the radar comprises a data transmission speed situation and/or a transmission initiating frequency parameter of the radar; and

determining, according to the data transmission situation, a feature parameter threshold corresponding to a basic body feature parameter of the target object.

Disclosed as a second aspect in the present disclosure is an apparatus for detecting a body feature parameter by a radar based on local computing, the apparatus including:

a compression distillation module, configured to perform, via the cloud, a compression distillation operation on a pre-trained basic computational model to obtain a to-be-deployed model corresponding to the basic computational model;

a deployment module, configured to deploy the to-be-deployed model to a device terminal corresponding to the radar to obtain a post-deployment model corresponding to the device terminal;

a receiving module, configured to receive a target parameter of a target object collected by the radar;

a judging module, configured to judge whether the target parameter satisfies a preset condition for computing a body feature parameter;

a computing module, configured to compute, in response to a judgment result of YES by the judging module, by the post-deployment model and the target parameter, a target body feature parameter of a target object, in which the target parameter of the target object comprises a type parameter, a distance parameter, a body movement parameter, and a basic body feature parameter of the target object.

As an optional implementation, in the second aspect of the present disclosure, the apparatus further includes:

a predicting module, configured to predict a detection scenario parameter of the radar before the compression distillation module performs, via the cloud, a compression distillation operation on a pre-trained basic computational model to obtain a to-be-deployed model corresponding to the basic computational model;

a determining module, configured to determine, according to the detection scenario parameter, a computational demand parameter of the device terminal corresponding to the radar, wherein the computational demand parameter comprises a computational volume demand parameter and/or a computational result demand parameter;

an acquiring module, configured to acquire a resource utilization parameter of the device terminal;

the determining module is further configured to determine, according to the resource utilization parameter, an operational performance parameter of the device terminal, in which the operational performance parameter includes a response duration parameter and/or an operational energy consumption parameter, and to determine, according to the computational demand parameter and the operational performance parameter, the compression distillation demand parameter corresponding to the pre-trained basic computational model;

the steps of the compression distillation module performing, via the cloud, a compression distillation operation on a pre-trained basic computational model to obtain a to-be-deployed model corresponding to the basic computational model include the followings in detail:

performing, via the cloud and the compression distillation demand parameter, a compression distillation operation on the basic computational model to obtain a to-be-deployed model corresponding to the basic computational model.

As an optional implementation, in the second aspect of the present disclosure, the determining module is further configured to

determine a model parameter of the to-be-deployed model before the deployment module deploys the to-be-deployed model to a device terminal corresponding to the radar, and obtains a post-deployment model corresponding to the device terminal, in which the model parameter includes at least one of a model size parameter, a model computational volume parameter, a model update manner parameter, and a framework compatibility requirement parameter;

the acquiring module is further configured to acquire a deployment environment parameter of the device terminal, in which the deployment environment parameter includes a deployment network parameter and/or a deployment system parameter;

the determining module is further configured to determine, according to the model parameter, the deployment environment parameter, and the operational performance parameter, a deployment parameter corresponding to the to-be-deployed model, in which the deployment parameter includes a deployment location parameter and/or a deployment time parameter;

the steps of the deployment module deploying the to-be-deployed model to a device terminal corresponding to the radar to obtain a post-deployment model corresponding to the device terminal include the followings in detail:

deploying, according to the deployment parameter, the to-be-deployed model to a device terminal corresponding to the radar to obtain a post-deployment model corresponding to the device terminal.

As an optional implementation, in the second aspect of the present disclosure, the acquiring module is further configured to

acquire a first radar parameter of the radar before the determining module determines, according to the model parameter, the deployment environment parameter, and the operational performance parameter, a deployment parameter corresponding to the to-be-deployed model, in which the first radar parameter includes at least one of a radar sampling rate parameter, a radar resolution parameter, and a radar data processing requirement parameter;

the determining module is further configured to determine, according to the first radar parameter, the deployment environment parameter, and the operational performance parameter, a deployment impact situation caused by the first radar parameter on the to-be-deployed model, and determining a deployment impact degree value corresponding to the deployment impact situation;

the judging module is further configured to judge whether the deployment impact degree value is greater than or equal to a preset deployment impact level threshold; the determining module determines, in response to a judgment result of NO by the judging module, according to the model parameter, the deployment environment parameter, and the operational performance parameter, a deployment parameter corresponding to the to-be-deployed model;

the determining module is further configured to determine, in response to a judgment result of YES by the judging module, according to the first radar parameter, the model parameter, the deployment environment parameter, and the operational performance parameter, a deployment parameter corresponding to the to-be-deployed model.

As an optional implementation, in the second aspect of the present disclosure, the target body feature parameter includes at least a blood pressure parameter, and the body movement parameter includes a body movement type parameter and a body movement amplitude parameter.

The steps of the judging module judging whether the target parameter satisfies a preset condition for computing a body feature parameter include the followings in detail:

judging, according to a type parameter of the target object, whether the type parameter is a preset type parameter;

judging, in response to a judgment of the type parameter being the preset type parameter, according to the distance parameter of the target object, whether the distance parameter is within a preset collecting range;

judging, in response to a judgment of the distance parameter being within the preset collecting range, according to the body movement type parameter of the target object, whether the body movement type parameter is a preset body movement type parameter;

judging, in response to a judgment of the body movement type parameter being the preset body movement type parameter, according to the body movement amplitude parameter of the target object, whether the body movement amplitude parameter is within a preset body movement amplitude range;

confirming, in response to a judgment of the body movement amplitude parameter being within a preset body movement amplitude range, that the target parameter satisfies the preset condition for computing a body feature parameter.

As an optional implementation, in the second aspect of the present disclosure, the steps of the judging module judging whether the target parameter satisfies a preset condition for computing a body feature parameter further include the followings in detail:

determining a first feature parameter and a second feature parameter corresponding to a basic body feature parameter of the target object before the confirming that the target parameter satisfies the preset condition for computing a body feature parameter, in which the first feature parameter includes a collecting time parameter and/or a collecting point number parameter, and the second feature parameter includes a waveform feature parameter;

judging whether the first feature parameter is greater than or equal to a preset feature parameter threshold;

determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold;

confirming, in response to a judgment of the waveform integrity being greater than or equal to a preset waveform integrity threshold, that the target parameter satisfies the preset condition for computing a body feature parameter.

As an optional implementation, in the second aspect of the present disclosure, the feature parameter threshold includes a collecting time threshold and/or a collecting point number threshold.

The feature parameter threshold is determined by following steps:

acquiring a second radar parameter of the radar, in which the second radar parameter of the radar comprises at least one of a radar sampling rate parameter, a radar resolution parameter, a radar data generating speed parameter, and a communication interface performance parameter of the radar;

determining, according to the second radar parameter, a data transmission situation of the radar, wherein the data transmission situation of the radar comprises a data transmission speed situation and/or a transmission initiating frequency parameter of the radar; and

determining, according to the data transmission situation, a feature parameter threshold corresponding to a basic body feature parameter of the target object.

Disclosed as a third aspect in the present disclosure is an apparatus for detecting a body feature parameter by a radar based on local computing, the apparatus including:

a memory, memorized with an executable code; and

a processor, coupled with the memory,

in which the processor invokes the executable code memorized in the memory to perform the method for detecting a body feature parameter by a radar based on local computing disclosed in the first aspect of the present disclosure.

Disclosed as a fourth aspect in the present disclosure is a non-transitory computer memory medium, and the non-transitory computer memory medium memorizes computer instructions; when the computer instructions are invoked, the method for detecting a body feature parameter by a radar based on local computing disclosed in the first aspect of the present disclosure is performed.

Compared to the prior art, the embodiments of the present disclosure have beneficial effects as follows.

According to the embodiments of the present disclosure, a compression distillation operation is performed on the basic computational model through the cloud to obtain the to-be-deployed model corresponding to the basic computational model, and deployed to the corresponding device terminal of the radar to obtain the corresponding post-deployment model at the device terminal; the target parameter of the target object collected by the radar is received, and it is judged whether the target parameter satisfies the conditions for computing a body feature parameter, and the target body feature parameter of the target object is computed, in response to a judgment result of YES, by the post-deployment model and the target parameter. Evidently, the implementation of the present disclosure enables the computation of the target body feature parameter of the target object by means of the post-deployment model deployed at the device terminal and the target parameter collected by the radar, such that the offline use of the device terminal is achieved when the radar detects the body feature parameter, thereby improving the real-time nature of the data transmission and the convenience of the data detection, and thus improving the detection efficiency of the radar on the body feature parameter.

### BRIEF DESCRIPTION OF THE DRAWINGS

In order to illustrate the technical solutions of the embodiments of the present disclosure more clearly, the following drawings are briefly described as required in the context of the embodiments. Obviously, the following drawings illustrate only some of the embodiments of the present disclosure. Other relevant drawings may be obtained on the basis of these drawings without any creative effort by those skilled in the art.

FIG. 1 is a schematic diagram of a technical framework for detecting a body feature parameter by a radar based on local computing as disclosed by an embodiment of the present disclosure;

FIG. 2 is a schematic flow diagram of a method for detecting a body feature parameter by a radar based on local computing as disclosed by an embodiment of the present disclosure;

FIG. 3 is a schematic flow diagram of another method for detecting a body feature parameter by a radar based on local computing as disclosed by an embodiment of the present disclosure;

FIG. 4 is a schematic structural diagram of an apparatus for detecting a body feature parameter by a radar based on local computing as disclosed by an embodiment of the present disclosure;

FIG. 5 is a schematic structural diagram of another apparatus for detecting a body feature parameter by a radar based on local computing as disclosed by an embodiment of the present disclosure;

FIG. 6 is a schematic structural diagram of yet another apparatus for detecting a body feature parameter by a radar based on local computing as disclosed by an embodiment of the present disclosure;

### DETAILED DESCRIPTION

For a better understanding of the solutions of the present disclosure by those skilled in the art, the technical solutions in the embodiments of the present disclosure are clearly and completely described and discussed below in conjunction with the attached drawings of the embodiments of the present disclosure. Obviously, the embodiments described herein are only some of the embodiments of the present disclosure but not all of them. Based on the embodiments in the present disclosure, all other embodiments acquired by those skilled in the art without inventive effort fall within the scope of protection of the present disclosure.

The terms “first”, “second”, and the like in the specification, the claims and the above-mentioned drawings of the present disclosure are used to identify different objects and are not intended to describe a particular sequence. In addition, the terms “comprise” and “include”, and any derivatives and conjugations thereof, are intended to cover non-exclusive inclusion. For example, a process, method, apparatus, product, or device that includes a series of steps or units is not limited to the listed steps or units, but optionally also includes steps or units that are not listed, or optionally also includes other steps or units that are inherent to those processes, methods, products, or devices.

The term “embodiment” herein means that a particular feature, structure or characteristic described in conjunction with an embodiment may be included in at least one embodiment of the present disclosure. The presence of the term in various places in the specification does not necessarily indicate the same embodiment, nor is it a separate or alternative embodiment that is mutually exclusive with other embodiments. It is understood, both explicitly and implicitly, by those skilled in the art that the embodiments described herein may be combined with other embodiments.

Disclosed in the present disclosure is a method for detecting a body feature parameter by a radar based on local computing and an apparatus therefor. The offline use of the device terminal is achieved when the radar detects the body feature parameter, thereby improving the real-time nature of the data transmission and the convenience method and the device and thus improving the detection efficiency of the radar on the body feature parameter.

### EMBODIMENT 1

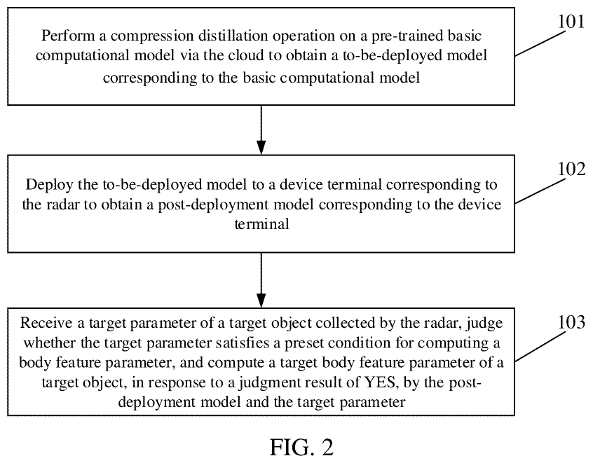

Referring to FIG. 2, FIG. 2 is a schematic flow diagram of a method for detecting a body feature parameter by a radar based on local computing as disclosed by an embodiment of the present disclosure. The method for a detecting body feature parameter by a radar based on local computing described in FIG. 2 may be applied to detecting a target body feature parameter (e.g., a blood pressure parameter-systolic/diastolic, heartbeat parameter, or respiratory parameter) by a radar of a target human body or a target animal. The detected target object and target body feature parameter types are not limited by the embodiments of the present disclosure. Optionally, the method may be achieved by a body feature parameter detecting device, which may be integrated in a body feature parameter detecting apparatus (e.g., a smart computer or smart phone), or a local server for processing the body feature parameter detecting process, which is not limited by the embodiments of the present disclosure. As shown in FIG. 2, the method for detecting a body feature parameter by a radar based on local computing may include the following steps:
Step 101: a compression distillation operation is performed on a pre-trained basic computational model via the cloud to obtain a to-be-deployed model corresponding to the basic computational model.

In the present embodiment of the disclosure, optionally, the compression distillation operation includes at least one of distillation, quantization, pruning, and low-rank decomposition.

Step 102: the to-be-deployed model is deployed to a device terminal corresponding to the radar to obtain a post-deployment model corresponding to the device terminal.

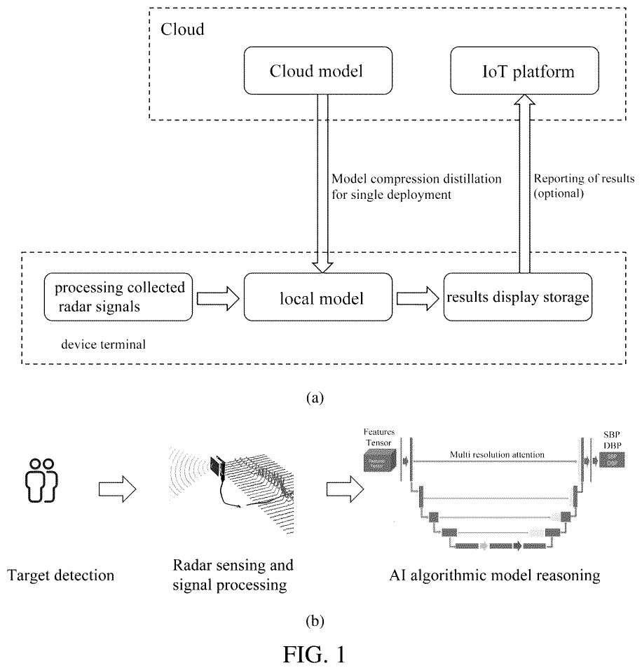

According to the present embodiment of the disclosure, as shown in inset (a) of FIG. 1, after the basic computational model (the cloud model) is trained in the cloud, the compression distillation operation can be carried out on the basic computational model, so that the obtained to-be-deployed model can be deployed to the corresponding device terminal of the radar as a post-deployment model, i.e., a local model. In this way, the post-deployment model can subsequently be used to complete, even when in the offline state, a target parameter calculation process for a target object collected by a radar.

Step 103: a target parameter of a target object collected by the radar is received, and it is judged whether the target parameter satisfies a preset condition for computing a body feature parameter, and a target body feature parameter of a target object is computed, in response to a judgment result of YES, by the post-deployment model and the target parameter.

According to the present embodiment of the disclosure, optionally, the target parameter of the target object includes the target type parameter, distance parameter, body movement parameter, and basic body feature parameter (e.g., waveform data such as heartbeat and respiration collected by the radar). Further and optionally, the target body feature parameter may include a blood pressure parameter, and may also include a heartbeat parameter, a respiration parameter and the like. The body movement parameter includes a body movement type parameter (e.g., hands vertically lowered in a sedentary state, hands raised flat in an upright state, and the like) and a body movement amplitude parameter.

It should be noted that, as shown in inset (b) of FIG. 1, after the start of the radar master control software, the model file on the device terminal is loaded to initialize the radar into the detection mode, and then according to the present invention, the sensed millimeter-wave radar signals can be combined with the local AI algorithmic framework to complete the offline state of the target identification, human/animal body feature detection (respiration, heartbeat, blood pressure, etc.) and other functions, which means that it realizes a set of hardware product solutions that enable local target detection functions.

Further, after computing the target body feature parameter of the target object, it can be stored on the device terminal, or the target body feature parameter can be displayed to the users in need (in case of abnormalities of the target body feature parameter, timely feedback can be provided), and it can also be uploaded to the cloud through the IoT technology.

Evidently, the implementation of the present embodiment of the disclosure enables the computation of the target body feature parameter of the target object by means of the post-deployment model deployed at the device terminal and the target parameter collected by the radar, such that the offline use of the device terminal is achieved when the radar detects the body feature parameter, thereby improving the real-time nature of the data transmission and the convenience of the data detection, and thus improving the detection efficiency of the radar on the body feature parameter. Also, additional communication costs and latency overhead are reduced.

In an optional implementation, before the compression distillation operation being performed on the pre-trained basic computational model via the cloud to obtain the to-be-deployed model corresponding to the basic computational model in aforementioned step 101, the method further includes:

predicting a detection scenario parameter of the radar and determining, according to the detection scenario parameter, a computational demand parameter of the device terminal corresponding to the radar;

acquiring a resource utilization parameter of the device terminal, and determining, according to the resource utilization parameter, an operational performance parameter of the device terminal; and

determining, according to the computational demand parameter and the operational performance parameter, the compression distillation demand parameter corresponding to the pre-trained basic computational model.

Further, in this optional implementation, the compression distillation operation being performed on the pre-trained basic computational model via the cloud to obtain the to-be-deployed model corresponding to the basic computational model in aforementioned step 101 includes:

performing, via the cloud and the compression distillation demand parameter, a compression distillation operation on the basic computational model to obtain a to-be-deployed model corresponding to the basic computational model.

In this optional implementation, optionally, the computational demand parameter includes a computational volume demand parameter and/or a computational result demand parameter. Further and optionally, the operational performance parameter includes a response duration parameter and/or an operational energy consumption parameter.

For example, if a family member in a home scenario needs to be tested for blood pressure, the radar corresponding to the device terminal can be determined to be relatively low in computational demand, and then combined with the resource utilization parameter of the device terminal, such as the CPU occupancy rate, memory occupancy parameter, storage read/write speed, and so on, to determine the response time of the device terminal, the power consumption parameter, and the heat generation parameter, and so on. Thus, the corresponding compression distillation demand parameter of the basic computational model is determined, such as removing certain neurons in the neural network, the need to maintain a high floating-point number parameter, the need to decompose the weight matrix into the product of three low-rank matrices, to achieve the compression distillation operation of the basic computational model.

Evidently, this optional embodiment can determine, according to the detecting scenario parameter of the radar, the computational demand parameter of the corresponding device terminal of the radar; determine, according to the resource utilization parameter of the device terminal, the operational performance parameter of the device terminal; and then determine, according to the computational demand parameter and the operational performance parameter, the compression distillation demand parameter corresponding to the basic computational model, thereby performing the compression distillation of the basic computational model according to the compression distillation demand parameter. In this way, the reliability and accuracy of the compression distillation operation of the basic computational model can be improved, and the normal operation of the device at the subsequent device terminal during the computation can be ensured, so as to accurately carry out the computation of the target body feature parameter of the target object.

In another optional embodiment, before the deploying the to-be-deployed model to a device terminal corresponding to the radar to obtain a post-deployment model corresponding to the device terminal in aforementioned step 102, the method further includes:

determining a model parameter of the to-be-deployed model;

acquiring a deployment environment parameter of the device terminal; and

determining, according to the model parameter, the deployment environment parameter, and the operational performance parameter, a deployment parameter corresponding to the to-be-deployed model.

Further, in this optional implementation, the deploying the to-be-deployed model to a device terminal corresponding to the radar to obtain a post-deployment model corresponding to the device terminal in aforementioned step 102 includes:

deploying, according to the deployment parameter, the to-be-deployed model to a device terminal corresponding to the radar to obtain a post-deployment model corresponding to the device terminal.

In this optional implementation, optionally, the model parameter includes at least one of a model size parameter, a model computational volume parameter, a model update manner parameter (e.g., update frequency and update time), and a framework compatibility requirement parameter; and the deployment environment parameter includes a deployment network parameter and/or a deployment system parameter (e.g., operating systems supported on the device terminal, such as Linux, Windows, or embedded systems, or hardware configurations on the device terminal, such as CPU, GPU, memory, and storage). Further and optionally, the deployment parameter includes a deployment location parameter and/or a deployment time parameter (e.g., deploy when the apparatus is idle or when the load is low).

Evidently, this optional embodiment can determine the deployment parameter corresponding to the to-be-deployed model according to the model parameter of the to-be-deployed model and the deployment environment parameter of the device terminal, combining with the operation performance parameter of the device terminal, thereby achieving the deployment process of the to-be-deployed model. In this way, the reliability and accuracy of the deployment of the to-be-deployed model can be improved to ensure the normal operation of the device terminal after the model is deployed, which is conducive to improving the reliability, accuracy, and validity of the computation of the target body feature parameter of the target object.

In yet another optional embodiment, before determining, according to the model parameter, the deployment environment parameter, and the operational performance parameter, a deployment parameter corresponding to the to-be-deployed model in the aforementioned step, the method further includes:

Acquiring a first radar parameter of the radar;

determining, according to the first radar parameter, the deployment environment parameter, and the operational performance parameter, a deployment impact situation caused by the first radar parameter on the to-be-deployed model, and determining a deployment impact degree value corresponding to the deployment impact situation;

judging whether the deployment impact degree value is greater than or equal to a preset deployment impact level threshold;

determining, in response to a judgment result of NO, according to the model parameter, the deployment environment parameter, and the operational performance parameter, a deployment parameter corresponding to the to-be-deployed model;

determining, in response to a judgment result of YES, according to the first radar parameter, the model parameter, the deployment environment parameter, and the operational performance parameter, a deployment parameter corresponding to the to-be-deployed model.

In this optional implementation, optionally, the first radar parameter includes at least one of a radar sampling rate parameter (e.g., number of samples per second), a radar resolution parameter (e.g., distance resolution and angle resolution), and a radar data processing requirement parameter (e.g., data filtering, pulse interference rejection, and differential computation);

For example, in a millimeter-wave radar system, the sampling rate of the radar is 1 MHz and the resolution is 0.1 meters. When the data processing requirements include pulse interference rejection, combined with the deployment environment parameters and operational performance parameters of the device terminal, it can be analyzed that the high sampling rate and high resolution lead to a larger amount of input data, which requires higher requirements for the storage and computational capabilities of the device terminal (i.e., if the model deployment location is not appropriate, it may increase the computational burden of the device terminal, as well as the real-time response of the model). Then, it can be determined that the value of the deployment impact degree is greater than the preset deployment impact degree threshold, which is then necessary to further determine the deployment parameter corresponding to the to-be-deployed model based on the first radar parameter, to carry out reasonable deployment of the model (such as deploying it to the edge server, adjusting the ratio of CPU and memory allocation at the device terminal, etc.).

Evidently, this optional embodiment can further determine the deployment impact of the first radar parameter on the to-be-deployed model according to the first radar parameter of the radar, the deployment environment parameter of the device terminal, and the operation performance parameter. In the case of a more significant deployment impact, the deployment parameter corresponding to the to-be-deployed model is determined based on the first radar parameter, so as to ensure that the deployment of the model at the device terminal is highly compatible with the actual operational requirements of the radar, thereby improving the overall adaptability of the system. At the same time, the deployment time and location of the model at the device terminal can be optimized, thereby improving the real-time computation and reliability of the system with respect to the target body feature parameters.

### EMBODIMENT 2

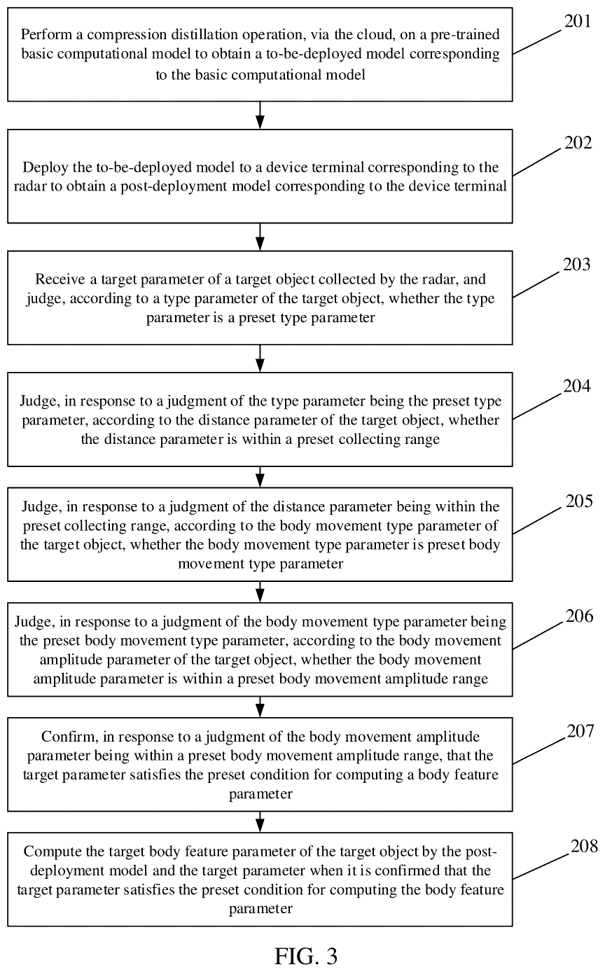

Referring to FIG. 3, FIG. 3 is a schematic flow diagram of another method for detecting a body feature parameter by a radar based on local computing as disclosed by an embodiment of the present disclosure. The method for a detecting body feature parameter by a radar based on local computing described in FIG. 3 may be applied to detecting a target body feature parameter (e.g., a blood pressure parameter-systolic/diastolic) by a radar of a target human body or a target animal. The detected target object and target body feature parameter types are not limited by the embodiments of the present disclosure. Optionally, the method may be achieved by a body feature parameter detecting device, which may be integrated in a body feature parameter detecting apparatus (e.g., a smart computer or smart phone), or a local server for processing the body feature parameter detecting process, which is not limited by the embodiments of the present disclosure. As shown in FIG. 3, the method for detecting a body feature parameter by a radar based on local computing may include the following steps:
Step 201: a compression distillation operation is performed, via the cloud, on a pre-trained basic computational model to obtain a to-be-deployed model corresponding to the basic computational model.

Step 202: the to-be-deployed model is deployed to a device terminal corresponding to the radar to obtain a post-deployment model corresponding to the device terminal.

Step 203: a target parameter of a target object collected by the radar is received, and it is judged, according to a type parameter of the target object, whether the type parameter is a preset type parameter;

Step 204: it is judged, in response to a judgment of the type parameter being the preset type parameter, according to the distance parameter of the target object, whether the distance parameter is within a preset collecting range;

Step 205: it is judged, in response to a judgment of the distance parameter being within the preset collecting range, according to the body movement type parameter of the target object, whether the body movement type parameter is preset body movement type parameter;

Step 206: it is judged, in response to a judgment of the body movement type parameter being the preset body movement type parameter, according to the body movement amplitude parameter of the target object, whether the body movement amplitude parameter is within a preset body movement amplitude range;

Step 207: it is confirmed, in response to a judgment of the body movement amplitude parameter being within a preset body movement amplitude range, that the target parameter satisfies the preset condition for computing a body feature parameter.

According to the present embodiment of the disclosure, for example, the judgment process of steps 203 to 207 can be understood as follows: firstly, it is judged whether the existence of the human body is true; if YES, it is judged whether the distance between the human body and the radar is in a range of 0.1 to 1 m; if YES, it is judged whether the human body is in a sedentary state; if YES, judging whether the body movement amplitude of the human body is less than a preset limit of the body movement amplitude of 20 (the greater the amplitude of the body movement is, the greater the human body movement is); if YES, then it can be confirmed that the target parameter collected by the radar satisfies the preset condition for computing the body feature parameter. Further, if any one of the judgment processes of steps 203 to 207 results in NO, it can be confirmed that the target parameter collected by the radar does not satisfy the preset condition for computing the body feature parameter.

Step 208: the target body feature parameter of the target object is computed by the post-deployment model and the target parameter when it is confirmed that the target parameter satisfies the preset condition for computing the body feature parameter.

In the present embodiment of the disclosure, regarding other descriptions of step 201, 202, and 208, please refer to the other detailed descriptions in embodiment 1 regarding step 101 to step 103, which is not repeated in the present embodiment of the disclosure.

Evidently, the implementation of the present embodiment of the disclosure can perform multiple parameter validity judgments on the target parameter of the target object collected by the radar, so that the processing of irrelevant targets or interfering data can be effectively reduced, which in turn can improve the computational efficiency and accuracy of the system. At the same time, it is also conducive to improving the adaptability and reliability of the system in complex scenarios and enhancing the user experience of the system.

In an optional implementation, before confirming that the target parameter satisfies the preset condition for computing a body feature parameter in aforementioned step 207, the method further includes:

determining a first feature parameter and a second feature parameter corresponding to a basic body feature parameter of the target object, and the second feature parameter includes a waveform feature parameter;

judging whether the first feature parameter is greater than or equal to a preset feature parameter threshold;

determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold;

confirming, in response to a judgment of the waveform integrity being greater than or equal to a preset waveform integrity threshold, that the target parameter satisfies the preset condition for computing a body feature parameter.

In this optional implementation, optionally, the first feature parameter includes a collecting time parameter and/or a collecting point number parameter. Further and optionally, the feature parameter threshold includes a collecting time threshold and/or a collecting point number threshold. For example, it can be understood as judging whether the basic body feature parameter of the target object collected by the radar has accumulated to a certain duration (e.g., 5 seconds of continuous collection) and/or the collection sample size; if YES, further judging whether the waveform of the basic body feature parameter (the parameter fed back by the radar is a waveform signal, such as the waveform data set of the heartbeat and the respiration in a preset period of time) is sufficiently complete; if YES, it can be confirmed that the target parameter satisfies the preset condition for computing the body feature parameter.

Further, the feature parameter threshold is determined by following steps:

acquiring a second radar parameter of the radar;

determining, according to the second radar parameter, a data transmission situation of the radar;

determining, according to the data transmission situation, a feature parameter threshold corresponding to a basic body feature parameter of the target object.

In this optional implementation, optionally, the second radar parameter of the radar includes at least one of a radar sampling rate parameter, a radar resolution parameter, a radar data generating speed parameter, and a communication interface performance parameter of the radar. Further and optionally, the data transmission situation of the radar includes a data transmission speed situation and/or a transmission initiating frequency parameter of the radar. In this way, it is ensured that data does not congest the radar firmware or cause the radar to initiate transmissions too frequently.

Evidently, this optional embodiment can effectively screen out high-quality data by judging a first feature parameter of a basic body feature parameter of a target object as well as analyzing the completeness of the waveform, reduce misjudgment or computational errors due to incomplete or insufficiently collected data, and thus can improve the computational reliability and accuracy of the target body feature parameter. At the same time, the dynamic determination of the feature parameter threshold through the second radar parameter of the radar can improve the flexibility of adjustment of the radar data collecting requirements, which in turn can ensure that the system can be operated efficiently on different radar equipment, and thus can enhance the adaptability and versatility of the system.

### EMBODIMENT 3

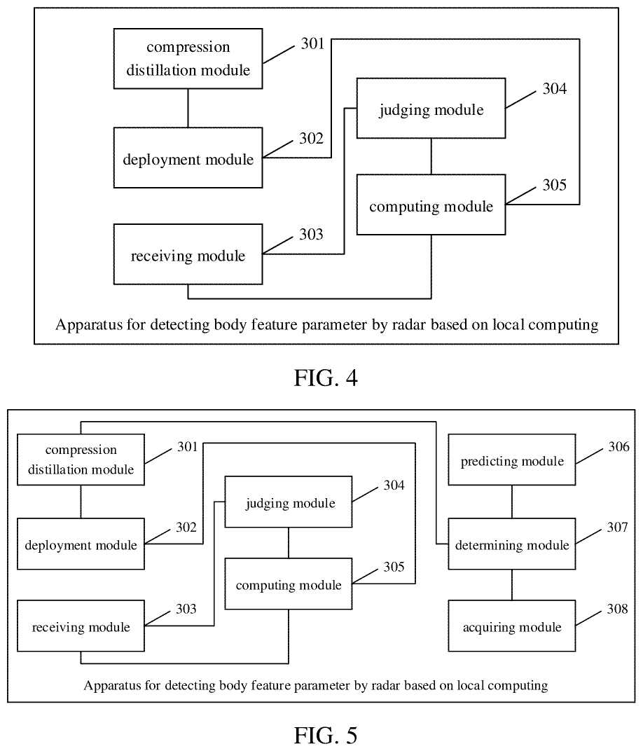

Referring to FIG. 4, FIG. 4 is a schematic structural diagram of an apparatus for detecting a body feature parameter by a radar based on local computing as disclosed by an embodiment of the present disclosure. As shown in FIG. 4, the apparatus for detecting a body feature parameter by a radar based on local computing may include:

a compression distillation module 301, configured to perform, via the cloud, a compression distillation operation on a pre-trained basic computational model to obtain a to-be-deployed model corresponding to the basic computational model;

a deployment module 302, configured to deploy the to-be-deployed model to a device terminal corresponding to the radar to obtain a post-deployment model corresponding to the device terminal;

a receiving module 303, configured to receive a target parameter of a target object collected by the radar;

a judging module 304, configured to judge whether the target parameter satisfies a preset condition for computing a body feature parameter; and

a computing module 305, configured to compute, in response to a judgment result of YES by the judging module 304, by the post-deployment model and the target parameter, a target body feature parameter of a target object.

In the present embodiment of the disclosure, the target parameter of the target object includes a type parameter, a distance parameter, a body movement parameter, and a basic body feature parameter of the target object.

Evidently, the implementation of the apparatus for detecting a body feature parameter by a radar based on local computing described by FIG. 4 enables the computation of the target body feature parameter of the target object by means of the post-deployment model deployed at the device terminal and the target parameter collected by the radar, such that the offline use of the device terminal is achieved when the radar detects the body feature parameter, thereby improving the real-time nature of the data transmission and the convenience of the data detection, and thus improving the detection efficiency of the radar on the body feature parameter. Also, additional communication costs and latency overhead are reduced.

In an optional implementation, the apparatus further includes:

a predicting module 306, configured to predict a detection scenario parameter of the radar before the compression distillation module 301 performs, via the cloud, a compression distillation operation on a pre-trained basic computational model to obtain a to-be-deployed model corresponding to the basic computational model;

a determining module 307, configured to determine, according to the detection scenario parameter, a computational demand parameter of the device terminal corresponding to the radar;

an acquiring module 308, configured to acquire a resource utilization parameter of the device terminal;

the determining module 307 is further configured to determine, according to the resource utilization parameter, an operational performance parameter of the device terminal, and to determine, according to the computational demand parameter and the operational performance parameter, the compression distillation demand parameter corresponding to the pre-trained basic computational model;

wherein the steps of the compression distillation module 301 performing, via the cloud, a compression distillation operation on a pre-trained basic computational model to obtain a to-be-deployed model corresponding to the basic computational model include the followings in detail:

performing, via the cloud and the compression distillation demand parameter, a compression distillation operation on the basic computational model to obtain a to-be-deployed model corresponding to the basic computational model.

In this optional implementation, the computational demand parameter includes a computational volume demand parameter and/or a computational result demand parameter; and the operational performance parameter includes a response duration parameter and/or an operational energy consumption parameter.

Evidently, the implementation of the apparatus for detecting a body feature parameter by a radar based on local computing described in FIG. 5 can determine, according to the detecting scenario parameter of the radar, the computational demand parameter of the corresponding device terminal of the radar; determine, according to the resource utilization parameter of the device terminal, the operational performance parameter of the device terminal; and then determine, according to the computational demand parameter and the operational performance parameter, the compression distillation demand parameter corresponding to the basic computational model, thereby performing the compression distillation of the basic computational model according to the compression distillation demand parameter. In this way, the reliability and accuracy of the compression distillation operation of the basic computational model can be improved, and the normal operation of the device at the subsequent device terminal during the computation can be ensured, so as to accurately carry out the computation of the target body feature parameter of the target object.

In another optional implementation, the determining module 307 is further configured to

determine a model parameter of the to-be-deployed model before the deployment module 302 deploys the to-be-deployed model to a device terminal corresponding to the radar to obtain a post-deployment model corresponding to the device terminal;

an acquiring module 308 is further configured to acquire a deployment environment parameter of the device terminal;

the determining module 307 is further configured to determine, according to the model parameter, the deployment environment parameter, and the operational performance parameter, a deployment parameter corresponding to the to-be-deployed model;

the steps of a deployment module 302 deploying the to-be-deployed model to a device terminal corresponding to the radar to obtain a post-deployment model corresponding to the device terminal include the followings in detail:

deploying, according to the deployment parameter, the to-be-deployed model to a device terminal corresponding to the radar to obtain a post-deployment model corresponding to the device terminal.

In this optional implementation, the model parameter includes at least one of a model size parameter, a model computational volume parameter, a model update manner parameter, and a framework compatibility requirement parameter; the deployment environment parameter includes a deployment network parameter and/or a deployment system parameter; and the deployment parameter includes a deployment location parameter and/or a deployment time parameter.

Evidently, the implementation of the apparatus for detecting a body feature parameter by a radar based on local computing described in FIG. 5 can determine the deployment parameter corresponding to the to-be-deployed model according to the model parameter of the to-be-deployed model and the deployment environment parameter of the device terminal, combining with the operation performance parameter of the device terminal, thereby achieving the deployment process of the to-be-deployed model. In this way, the reliability and accuracy of the deployment of the to-be-deployed model can be improved to ensure the normal operation of the device terminal after the model is deployed, which is conducive to improving the reliability, accuracy, and validity of the computation of the target body feature parameter of the target object.

In yet another optional implementation, the acquiring module 308 is further configured to

acquire a first radar parameter of the radar before the determining module 307 determines, according to the model parameter, the deployment environment parameter, and the operational performance parameter, a deployment parameter corresponding to the to-be-deployed model;

the determining module 307 is further configured to determine, according to the first radar parameter, the deployment environment parameter, and the operational performance parameter, a deployment impact situation caused by the first radar parameter on the to-be-deployed model, and determining a deployment impact degree value corresponding to the deployment impact situation;

the judging module 304 is further configured to judge whether the deployment impact degree value is greater than or equal to a preset deployment impact level threshold; the determining module 307 determines, in response to a judgment result of NO by the judging module 304, according to the model parameter, the deployment environment parameter, and the operational performance parameter, a deployment parameter corresponding to the to-be-deployed model;

the determining module 307 is further configured to determine, in response to a judgment result of YES by judging module 304, according to the first radar parameter, the model parameter, the deployment environment parameter, and the operational performance parameter, a deployment parameter corresponding to the to-be-deployed model.

In this optional implementation, the first radar parameter includes at least one of a radar sampling rate parameter, a radar resolution parameter, and a radar data processing requirement parameter.

Evidently, the implementation of the apparatus for detecting a body feature parameter by a radar based on local computing described in FIG. 5 can further determine the deployment impact of the first radar parameter on the to-be-deployed model according to the first radar parameter of the radar, the deployment environment parameter of the device terminal, and the operation performance parameter. In the case of a more significant deployment impact, the deployment parameter corresponding to the to-be-deployed model is determined based on the first radar parameter, so as to ensure that the deployment of the model at the device terminal is highly compatible with the actual operational requirements of the radar, thereby improving the overall adaptability of the system. At the same time, the deployment time and location of the model at the device terminal can be optimized, thereby improving the real-time computation and reliability of the system with respect to the target body feature parameters.

In yet another optional implementation, the target body feature parameter includes at least a blood pressure parameter, and the body movement parameter includes a body movement type parameter and a body movement amplitude parameter.

The steps of the judging module 304 judging whether the target parameter satisfies a preset condition for computing a body feature parameter include the followings in detail:

judging, according to a type parameter of the target object, whether the type parameter is a preset type parameter;

judging, in response to a judgment of the type parameter being the preset type parameter, according to the distance parameter of the target object, whether the distance parameter is within a preset collecting range;

judging, in response to a judgment of the distance parameter being within the preset collecting range, according to the body movement type parameter of the target object, whether the body movement type parameter is preset body movement type parameter;

judging, in response to a judgment of the body movement type parameter being the preset body movement type parameter, according to the body movement amplitude parameter of the target object, whether the body movement amplitude parameter is within a preset body movement amplitude range;

confirming, in response to a judgment of the body movement amplitude parameter being within a preset body movement amplitude range, that the target parameter satisfies the preset condition for computing a body feature parameter.

Evidently, the implementation of the apparatus for detecting a body feature parameter by a radar based on local computing described in FIG. 5 can perform multiple parameter validity judgments on the target parameter of the target object collected by the radar, so that the processing of irrelevant targets or interfering data can be effectively reduced, which in turn can improve the computational efficiency and accuracy of the system. At the same time, it is also conducive to improving the adaptability and reliability of the system in complex scenarios and enhancing the user experience of the system.

In yet another optional implementation, the steps of the judging module 304 judging whether the target parameter satisfies a preset condition for computing a body feature parameter further include the followings in detail:

determining a first feature parameter and a second feature parameter corresponding to a basic body feature parameter of the target object before confirming that the target parameter satisfies the preset condition for computing a body feature parameter, in which the second feature parameter includes a waveform feature parameter;

judging whether first feature parameter is greater than or equal to a preset feature parameter threshold;

determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold;

confirming, in response to a judgment of the waveform integrity being greater than or equal to a preset waveform integrity threshold, that the target parameter satisfies the preset condition for computing a body feature parameter.

In this optional implementation, the first feature parameter includes a collecting time parameter and/or a collecting point number parameter; and the feature parameter threshold includes a collecting time threshold and/or a collecting point number threshold.

Further, in this optional implementation, the feature parameter threshold is determined by the following steps:

acquiring a second radar parameter of the radar;

determining, according to the second radar parameter, a data transmission situation of the radar;

determining, according to the data transmission situation, a feature parameter threshold corresponding to a basic body feature parameter of the target object.

In this optional implementation, optionally, the second radar parameter of the radar includes at least one of a radar sampling rate parameter, a radar resolution parameter, a radar data generating speed parameter, and a communication interface performance parameter of the radar; and the data transmission situation of the radar includes a data transmission speed situation and/or a transmission initiating frequency parameter of the radar.

Evidently, the implementation of the apparatus for detecting a body feature parameter by a radar based on local computing described in FIG. 5 can effectively screen out high-quality data by judging a first feature parameter of a basic body feature parameter of a target object as well as analyzing the completeness of the waveform, reduce misjudgment or computational errors due to incomplete or insufficiently collected data, and thus can improve the computational reliability and accuracy of the target body feature parameter. At the same time, the dynamic determination of the feature parameter threshold through the second radar parameter of the radar can improve the flexibility of adjustment of the radar data collecting requirements, which in turn can ensure that the system can be operated efficiently on different radar equipment, and thus can enhance the adaptability and versatility of the system.

### EMBODIMENT 4

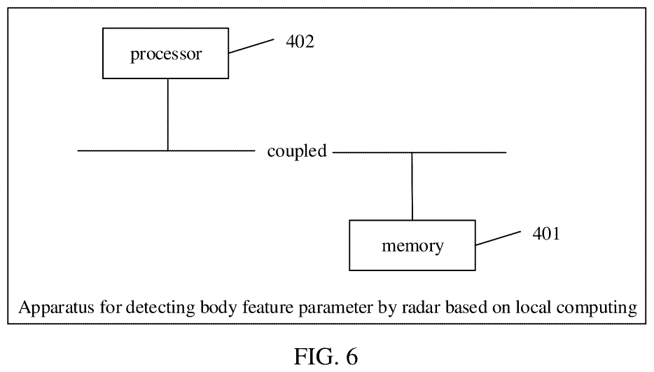

Referring to FIG. 6, FIG. 6 is a schematic structural diagram of yet another apparatus for detecting a body feature parameter by a radar based on local computing as disclosed by an embodiment of the present disclosure. As shown in FIG. 6, the apparatus for detecting a body feature parameter by a radar based on local computing may include:

a memory 401, memorized with an executable code; and

a processor 402, coupled with memory 401,

wherein the processor 402 invokes the executable code memorized in the memory 401 to perform steps of the method for detecting a body feature parameter by a radar based on local computing as described in the first embodiment of the present disclosure or the second embodiment of the present disclosure.

### EMBODIMENT 5

Disclosed in the present embodiment of the disclosure is a non-transitory computer memory medium, the non-transitory computer memory medium memorizes computer instructions; when the computer instructions are invoked, steps of the method for detecting a body feature parameter by a radar based on local computing described in the first embodiment and second embodiment of the present disclosure are performed.

### SIXTH EMBODIMENT

Disclosed in the present embodiment of the disclosure is a computer program product, the computer program product including a non-transitory computer readable memory medium memorized with a computer program. The computer program may be operated to enable the computer to perform steps in the method for detecting a body feature parameter by a radar based on local computing described in the first embodiment or second embodiment.

The aforementioned described embodiment of the apparatus is only illustrative. The modules described as separate components may or may not be physically separated, and the modules used as components for display may or may not be physical modules, that is, they may be located in the same place or may be distributed to a plurality of network modules. Some or all these modules may be selected according to practical demands to achieve the purpose of the solution of the present embodiment. It may be understood and performed by a person of ordinary skill in the art without inventive effort.

With the specific description of the above embodiments, it is clear to those skilled in the art that the various implementations may be implemented with the aid of software plus the necessary common hardware platform, and admittedly, with the aid of hardware. Based on such an understanding, the above technical solutions that essentially or contribute to the prior art may be embodied in the form of a software product which may be memorized in a non-transitory computer-readable memory medium, the non-transitory memory medium including Read-Only Memory, Random Access Memory, Programmable Read-only Memory, Erasable Programmable Read Only Memory, One-time Programmable Read-Only Memory, Electrically-Erasable Programmable Read-Only Memory, Compact Disc Read-Only Memory, other Compact Disc Memory, Disk Memory, Tape Memory or any other non-transitory computer-readable medium that may be used to carry or memorize data.

Finally, it should be noted that the method for detecting a body feature parameter by a radar based on local computing and the apparatus therefor disclosed in the embodiments of the present disclosure are only preferred embodiments of the present disclosure, and are only used to illustrate the technical solutions of the present disclosure, but not to limit them. Despite the detailed description of the disclosure with reference to the aforementioned embodiments, it should be understood, by those skilled in the art, that the technical solutions recorded in the aforementioned embodiments may still be modified, or equivalent substitutions for some of the technical features thereof may be made; which the essence of the corresponding technical solutions of these modifications or substitutions is without departing from the spirit and scope of the technical solutions of the various embodiments of the disclosure.

1. A method for detecting a body feature parameter by a radar based on local computing, wherein the method comprises:
performing, via the cloud, a compression distillation operation on a pre-trained basic computational model to obtain a to-be-deployed model corresponding to the basic computational model;
deploying the to-be-deployed model to a device terminal corresponding to the radar to obtain a post-deployment model corresponding to the device terminal; and
receiving a target parameter of a target object collected by the radar, and judging whether the target parameter satisfies a preset condition for computing a body feature parameter, and computing, in response to a judgment result of YES, by the post-deployment model and the target parameter, a target body feature parameter of a target object, wherein the target parameter of the target object comprises a type parameter, a distance parameter, a body movement parameter, and a basic body feature parameter of the target object.

2. The method for detecting a body feature parameter by a radar based on local computing according to claim 1, wherein, before the performing, via the cloud, the compression distillation operation on the pre-trained basic computational model to obtain the to-be-deployed model corresponding to the basic computational model, the method further comprises:
predicting a detection scenario parameter of the radar and determining, according to the detection scenario parameter, a computational demand parameter of the device terminal corresponding to the radar, wherein the computational demand parameter comprises a computational volume demand parameter and/or a computational result demand parameter;
acquiring a resource utilization parameter of the device terminal, and determining, according to the resource utilization parameter, an operational performance parameter of the device terminal, wherein the operational performance parameter comprises a response duration parameter and/or an operational energy consumption parameter; and
determining, according to the computational demand parameter and the operational performance parameter, the compression distillation demand parameter corresponding to the pre-trained basic computational model;
wherein the performing, via the cloud, a compression distillation operation on a pre-trained basic computational model to obtain a to-be-deployed model corresponding to the basic computational model comprises:
performing, via the cloud and the compression distillation demand parameter, a compression distillation operation on the basic computational model to obtain a to-be-deployed model corresponding to the basic computational model.

3. The method for detecting a body feature parameter by a radar based on local computing according to claim 2, wherein, before the deploying the to-be-deployed model to a device terminal corresponding to the radar to obtain a post-deployment model corresponding to the device terminal, the method further comprises:
determining a model parameter of the to-be-deployed model, wherein the model parameter comprises at least one of a model size parameter, a model computational volume parameter, a model update manner parameter, and a framework compatibility requirement parameter;
acquiring a deployment environment parameter of the device terminal, wherein the deployment environment parameter comprises a deployment network parameter and/or a deployment system parameter;
determining, according to the model parameter, the deployment environment parameter, and the operational performance parameter, a deployment parameter corresponding to the to-be-deployed model, wherein the deployment parameter comprises a deployment location parameter and/or a deployment time parameter;
wherein the deploying the to-be-deployed model to a device terminal corresponding to the radar to obtain a post-deployment model corresponding to the device terminal comprises:
deploying, according to the deployment parameter, the to-be-deployed model to a device terminal corresponding to the radar to obtain a post-deployment model corresponding to the device terminal.

4. The method for detecting a body feature parameter by a radar based on local computing according to claim 3, wherein, before the determining, according to the model parameter, the deployment environment parameter, and the operational performance parameter, a deployment parameter corresponding to the to-be-deployed model, the method further comprises:
acquiring a first radar parameter of the radar, wherein the first radar parameter comprises at least one of a radar sampling rate parameter, a radar resolution parameter, and a radar data processing requirement parameter;
determining, according to the first radar parameter, the deployment environment parameter, and the operational performance parameter, a deployment impact situation caused by the first radar parameter on the to-be-deployed model, and determining a deployment impact degree value corresponding to the deployment impact situation;
judging whether the deployment impact degree value is greater than or equal to a preset deployment impact level threshold;
determining, in response to a judgment result of NO, according to the model parameter, the deployment environment parameter, and the operational performance parameter, a deployment parameter corresponding to the to-be-deployed model;
determining, in response to a judgment result of YES, according to the first radar parameter, the model parameter, the deployment environment parameter, and the operational performance parameter, a deployment parameter corresponding to the to-be-deployed model.

5. The method for detecting a body feature parameter by a radar based on local computing according to claim 1, wherein the target body feature parameter comprises at least a blood pressure parameter, and the body movement parameter comprises a body movement type parameter and a body movement amplitude parameter;
wherein the judging whether the target parameter satisfies a preset condition for computing a body feature parameter comprises:
judging, according to a type parameter of the target object, whether the type parameter is a preset type parameter;
judging, in response to a judgment of the type parameter being the preset type parameter, according to the distance parameter of the target object, whether the distance parameter is within a preset collecting range;
judging, in response to a judgment of the distance parameter being within the preset collecting range, according to the body movement type parameter of the target object, whether the body movement type parameter is a preset body movement type parameter;
judging, in response to a judgment of the body movement type parameter being the preset body movement type parameter, according to the body movement amplitude parameter of the target object, whether the body movement amplitude parameter is within a preset body movement amplitude range; and
confirming, in response to a judgment of the body movement amplitude parameter being within a preset body movement amplitude range, that the target parameter satisfies the preset condition for computing a body feature parameter.

6. The method for detecting a body feature parameter by a radar based on local computing according to claim 2, wherein the target body feature parameter comprises at least a blood pressure parameter, and the body movement parameter comprises a body movement type parameter and a body movement amplitude parameter;
wherein the judging whether the target parameter satisfies a preset condition for computing a body feature parameter comprises:
judging, according to a type parameter of the target object, whether the type parameter is a preset type parameter;
judging, in response to a judgment of the type parameter being the preset type parameter, according to the distance parameter of the target object, whether the distance parameter is within a preset collecting range;
judging, in response to a judgment of the distance parameter being within the preset collecting range, according to the body movement type parameter of the target object, whether the body movement type parameter is a preset body movement type parameter;
judging, in response to a judgment of the body movement type parameter being the preset body movement type parameter, according to the body movement amplitude parameter of the target object, whether the body movement amplitude parameter is within a preset body movement amplitude range; and
confirming, in response to a judgment of the body movement amplitude parameter being within a preset body movement amplitude range, that the target parameter satisfies the preset condition for computing a body feature parameter.

7. The method for detecting a body feature parameter by a radar based on local computing according to claim 3, wherein the target body feature parameter comprises at least a blood pressure parameter, and the body movement parameter comprises a body movement type parameter and a body movement amplitude parameter;
wherein the judging whether the target parameter satisfies a preset condition for computing a body feature parameter comprises:
judging, according to a type parameter of the target object, whether the type parameter is a preset type parameter;
judging, in response to a judgment of the type parameter being the preset type parameter, according to the distance parameter of the target object, whether the distance parameter is within a preset collecting range;
judging, in response to a judgment of the distance parameter being within the preset collecting range, according to the body movement type parameter of the target object, whether the body movement type parameter is a preset body movement type parameter;
judging, in response to a judgment of the body movement type parameter being the preset body movement type parameter, according to the body movement amplitude parameter of the target object, whether the body movement amplitude parameter is within a preset body movement amplitude range; and
confirming, in response to a judgment of the body movement amplitude parameter being within a preset body movement amplitude range, that the target parameter satisfies the preset condition for computing a body feature parameter.

8. The method for detecting a body feature parameter by a radar based on local computing according to claim 4, wherein the target body feature parameter comprises at least a blood pressure parameter, and the body movement parameter comprises a body movement type parameter and a body movement amplitude parameter;
wherein the judging whether the target parameter satisfies a preset condition for computing a body feature parameter comprises:
judging, according to a type parameter of the target object, whether the type parameter is a preset type parameter;
judging, in response to a judgment of the type parameter being the preset type parameter, according to the distance parameter of the target object, whether the distance parameter is within a preset collecting range;
judging, in response to a judgment of the distance parameter being within the preset collecting range, according to the body movement type parameter of the target object, whether the body movement type parameter is a preset body movement type parameter;
judging, in response to a judgment of the body movement type parameter being the preset body movement type parameter, according to the body movement amplitude parameter of the target object, whether the body movement amplitude parameter is within a preset body movement amplitude range; and
confirming, in response to a judgment of the body movement amplitude parameter being within a preset body movement amplitude range, that the target parameter satisfies the preset condition for computing a body feature parameter.

9. The method for detecting a body feature parameter by a radar based on local computing according to claim 5, wherein, before the confirming that the target parameter satisfies the preset condition for computing a body feature parameter, the method further comprises:
determining a first feature parameter and a second feature parameter corresponding to a basic body feature parameter of the target object, wherein the first feature parameter comprises a collecting time parameter and/or a collecting point number parameter, and wherein the second feature parameter comprises a waveform feature parameter;
judging whether the first feature parameter is greater than or equal to a preset feature parameter threshold;
determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold; and
confirming, in response to a judgment of the waveform integrity being greater than or equal to a preset waveform integrity threshold, that the target parameter satisfies the preset condition for computing a body feature parameter.

10. The method for detecting a body feature parameter by a radar based on local computing according to claim 6, wherein, before the confirming that the target parameter satisfies the preset condition for computing a body feature parameter, the method further comprises:
determining a first feature parameter and a second feature parameter corresponding to a basic body feature parameter of the target object, wherein the first feature parameter comprises a collecting time parameter and/or a collecting point number parameter, and wherein the second feature parameter comprises a waveform feature parameter;
judging whether the first feature parameter is greater than or equal to a preset feature parameter threshold;
determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold; and
confirming, in response to a judgment of the waveform integrity being greater than or equal to a preset waveform integrity threshold, that the target parameter satisfies the preset condition for computing a body feature parameter.

11. The method for detecting a body feature parameter by a radar based on local computing according to claim 7, wherein, before the confirming that the target parameter satisfies the preset condition for computing a body feature parameter, the method further comprises:
determining a first feature parameter and a second feature parameter corresponding to a basic body feature parameter of the target object, wherein the first feature parameter comprises a collecting time parameter and/or a collecting point number parameter, and wherein the second feature parameter comprises a waveform feature parameter;
judging whether the first feature parameter is greater than or equal to a preset feature parameter threshold;
determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold; and
confirming, in response to a judgment of the waveform integrity being greater than or equal to a preset waveform integrity threshold, that the target parameter satisfies the preset condition for computing a body feature parameter.

12. The method for detecting a body feature parameter by a radar based on local computing according to claim 8, wherein, before the confirming that the target parameter satisfies the preset condition for computing a body feature parameter, the method further comprises:
determining a first feature parameter and a second feature parameter corresponding to a basic body feature parameter of the target object, wherein the first feature parameter comprises a collecting time parameter and/or a collecting point number parameter, and wherein the second feature parameter comprises a waveform feature parameter;
judging whether the first feature parameter is greater than or equal to a preset feature parameter threshold;
determining, in response to a judgment of the first feature parameter being greater than or equal to the preset feature parameter threshold, according to the waveform feature parameter, a waveform integrity corresponding to the basic body feature parameter, and judging whether the waveform integrity is greater than or equal to a preset waveform integrity threshold; and
confirming, in response to a judgment of the waveform integrity being greater than or equal to a preset waveform integrity threshold, that the target parameter satisfies the preset condition for computing a body feature parameter.

13. The method for detecting a body feature parameter by a radar based on local computing according to claim 9, wherein the feature parameter threshold comprises a collecting time threshold and/or a collecting point number threshold;
wherein the feature parameter threshold is determined by following steps:
acquiring a second radar parameter of the radar, wherein the second radar parameter of the radar comprises at least one of a radar sampling rate parameter, a radar resolution parameter, a radar data generating speed parameter, and a communication interface performance parameter of the radar;
determining, according to the second radar parameter, a data transmission situation of the radar, wherein the data transmission situation of the radar comprises a data transmission speed situation and/or a transmission initiating frequency parameter of the radar; and
determining, according to the data transmission situation, a feature parameter threshold corresponding to a basic body feature parameter of the target object.

14. The method for detecting a body feature parameter by a radar based on local computing according to claim 10, wherein the feature parameter threshold comprises a collecting time threshold and/or a collecting point number threshold;
wherein the feature parameter threshold is determined by following steps:
acquiring a second radar parameter of the radar, wherein the second radar parameter of the radar comprises at least one of a radar sampling rate parameter, a radar resolution parameter, a radar data generating speed parameter, and a communication interface performance parameter of the radar;
determining, according to the second radar parameter, a data transmission situation of the radar, wherein the data transmission situation of the radar comprises a data transmission speed situation and/or a transmission initiating frequency parameter of the radar; and
determining, according to the data transmission situation, a feature parameter threshold corresponding to a basic body feature parameter of the target object.

15. The method for detecting a body feature parameter by a radar based on local computing according to claim 11, wherein the feature parameter threshold comprises a collecting time threshold and/or a collecting point number threshold;
wherein the feature parameter threshold is determined by following steps:
acquiring a second radar parameter of the radar, wherein the second radar parameter of the radar comprises at least one of a radar sampling rate parameter, a radar resolution parameter, a radar data generating speed parameter, and a communication interface performance parameter of the radar;
determining, according to the second radar parameter, a data transmission situation of the radar, wherein the data transmission situation of the radar comprises a data transmission speed situation and/or a transmission initiating frequency parameter of the radar; and
determining, according to the data transmission situation, a feature parameter threshold corresponding to a basic body feature parameter of the target object.

16. The method for detecting a body feature parameter by a radar based on local computing according to claim 12, wherein the feature parameter threshold comprises a collecting time threshold and/or a collecting point number threshold;
wherein the feature parameter threshold is determined by following steps:
acquiring a second radar parameter of the radar, wherein the second radar parameter of the radar comprises at least one of a radar sampling rate parameter, a radar resolution parameter, a radar data generating speed parameter, and a communication interface performance parameter of the radar;
determining, according to the second radar parameter, a data transmission situation of the radar, wherein the data transmission situation of the radar comprises a data transmission speed situation and/or a transmission initiating frequency parameter of the radar; and
determining, according to the data transmission situation, a feature parameter threshold corresponding to a basic body feature parameter of the target object.

17. An apparatus for detecting a body feature parameter by a radar based on local computing, wherein the apparatus comprises:
a compression distillation module, configured to perform, via the cloud, a compression distillation operation on a pre-trained basic computational model to obtain a to-be-deployed model corresponding to the basic computational model;
a deployment module, configured to deploy the to-be-deployed model to a device terminal corresponding to the radar to obtain a post-deployment model corresponding to the device terminal;
a receiving module, configured to receive a target parameter of a target object collected by the radar;
a judging module, configured to judge whether the target parameter satisfies a preset condition for computing a body feature parameter; and
a computing module, configured to compute, in response to a judgment result of YES by the judging module, by the post-deployment model and the target parameter, a target body feature parameter of a target object, wherein the target parameter of the target object comprises a type parameter, a distance parameter, a body movement parameter, and a basic body feature parameter of the target object.

18. An apparatus for detecting a body feature parameter by a radar based on local computing, wherein the apparatus comprises:
a memory, memorized with an executable code; and
a processor, coupled with the memory,
wherein the processor invokes the executable code memorized in the memory to perform a method for detecting a body feature parameter by a radar based on local computing, wherein the method comprises:
performing, via the cloud, a compression distillation operation on a pre-trained basic computational model to obtain a to-be-deployed model corresponding to the basic computational model;
deploying the to-be-deployed model to a device terminal corresponding to the radar to obtain a post-deployment model corresponding to the device terminal; and
receiving a target parameter of a target object collected by the radar, and judging whether the target parameter satisfies a preset condition for computing a body feature parameter, and computing, in response to a judgment result of YES, by the post-deployment model and the target parameter, a target body feature parameter of a target object, wherein the target parameter of the target object comprises a type parameter, a distance parameter, a body movement parameter, and a basic body feature parameter of the target object.
